(19) United States
(12) Patent Application Publication
(10) Pub. No.: US 2003/0093017 A1
(43) Pub. Date: May 15, 2003

(54) Wrist wrap

(76) Inventor: Annette M. Loud, Detroit, MI (US)

Correspondence Address: Annette M. Loud, 219 Hague Street, Detroit, MI, 48202, US

(21) Appl. No.: 09/843,971

(22) Filed: Apr. 27, 2001

(51) Int. Cl. A61F 5/00

(52) U.S. Cl. 602/2; 602/21

(57) ABSTRACT

A therapeutic joint wrap device for strained, sprained, or inflamed wrists has a triangularly shaped piece of material having a truncated apex with an elastic piece of material attached to the truncated apex at its corners and a base that is attached to a rectangularly shaped piece of material at one end on the longest dimensional side where the rectangularly shaped piece of material is of a sufficient length to completely encircle a person's wrist. This device also has a smaller piece of material attached to the rectangularly shaped piece of material on three sides thereof to form a pocket on one side thereof for receiving either a heating pack or a cold pack therein, and attaching means for securing the device around a person's wrist, and an elastic band attached to the base of the triangularly shaped piece of material.

### FIELD OF THE INVENTION

The present invention relates to a therapeutic joint wrap for strained or sprained joints. More specifically, it relates to a wrap device for a strained or sprained or inflamed wrist

### BACKGROUND OF THE INVENTION

Prior to the present invention, strained or sprained or inflamed wrist joints were treated with heat or ice wrapped with large bulky bandages that were also used on other parts of the body. These bandages were awkward and unsightly and cumbersome to use because they were not designed for this joint. Over the years, more and more people have become afflicted with problems of the wrists, not only athletes but also industrial plant and office workers who perform tasks that put a stress on the wrist. Examples of these types of jobs are people who sit behind a computer all day using the keyboard as well as assembly line workers in a plant who perform repetitive wrist movement type jobs that places stress on the wrist.

Currently, the devices that are used in the field of sports and rehabilitative medical applications are plain wrap bandages, gloves, braces, etc., which are used to stabilize and support an injury. Quite often an athlete can be seen with tape and/or a flexible bandage heavily wrapped around the wrist. In some instances this device almost renders the injured hand impractical to use because of the bandages. If an ice pack or heating pad is needed for the injured wrist, in the case of ice, the bulky wrapped wist with ice is heavy, awkward and messy to use due to melting. It does not easily conform to the shape of the wrist and the ice quickly melts. In the case of a thermal compress, when the conventional heating pad is removed from the external source of heat, it quickly returns to ambient temperatures.

As mentioned above, many individuals suffer from chronic discomfort in their mists due to muscle injury, tendinitis, or permanent ligament damage. Since it appears that more and more people are injuring their wrist, such as carpal tunnel syndrome, because of the deficiencies of the work places, there is a need for a medical device for allowing a person with an injury of the ones mentioned above (i.e., due to strains and/or sprains) the ability to continue to perform his/her work while nursing that minor injury.

### SUMMARY OF THE INVENTION

The present invention is directed to a therapeutic wrist wrap device comprising a triangularly shaped piece of material having a truncated apex with an elastic piece of material attached to the truncated apex at its corners and a base that is attached to a rectangularly shaped piece of material at one end on the longest dimensional side where the rectangularly shaped piece of material is of a sufficient length to completely encircle a person's wrist, has a smaller piece of material attached to the rectangularly shaped piece on three sides thereof to form a pocket on one side thereof for receiving either a heating pack or a cold pack therein, and attaching means for securing the device around a person's wrist, and an elastic band attached to the base of the triangularly shaped piece of material.

### BRIEF DESCRIPTION OF THE DRAWINGS

FIG. 1 is a plan view of the wrist wrap device showing the bottom side of the wrist wrap device.

FIG. 2 is a perspective view of the wrist wrap device showing a side view.

FIG. 3 is a plan view showing the topside of the device.

FIG. 4 is a top view with the wrist wrap in place on a hand.

### DETAILED DESCRIPTION OF THE INVENTION

It was surprisingly found that a tailored wrist wrap device could be developed that is functional yet effective for the intended use. Its appearance in place is not unsightly and some times unnoticeable and yet gives the users as much function out of the wrist as he/she needs for performing daily tasks. Such device is lightweight and aids healing by being able to be used in a wide range of tasks from completely immobilizing the wrist to providing many degrees of flexibility or range of motion. A person using this device can have his/her personal tailored plan for bringing him/her back slowly and effectively for a complete recovery.

One of the things that makes this wrist wrap so versatile is that it is able to use a hot or cold pack in the specially designed pocket in the wrap without making a wet mess with the cold pack; it also incorporates slats commonly referred to as “bones” for immobilizing the wrist completely. The wrap itself lends comfort and support, is very light weight, and more importantly, is easily adjustable by the wearer.

Referring now to the drawings, FIG. 1 shows the complete device (100) of the present invention from a plan view from the bottom side of the device with its component parts. The components of the device are a main rectangularly shaped piece of material (1) that has a triangularly shaped piece of material (2) that has a truncated apex (3) wherein the triangularly shaped piece of material (2) is attached at its base to one end of the rectangularly shaped piece of material (1) on its longest dimensional side. The rectangularly shaped piece of material (1) is of a sufficient length to completely encircle a person's wrist. At the same end of the rectangularly shaped piece of material as where the triangularly shaped piece of material (2) is attached a smaller piece of material is attached on three sides to form a pocket (4) for receiving and holding a cold or heating pack therein. A piece of elastic material (5), the length of a diameter of a person's finger is attached to each side of the truncated apex (3). Another piece of elastic material (6) is attached below the intersection where the base of the triangularly shaped piece of material (2) is attached to the rectangularly shaped piece of material (1). Elastic material (6) is of a sufficient length as to securely hold the device to a person's wrist. This assists in placing the device on the wrist and in the wearer's ability to adjust the device.

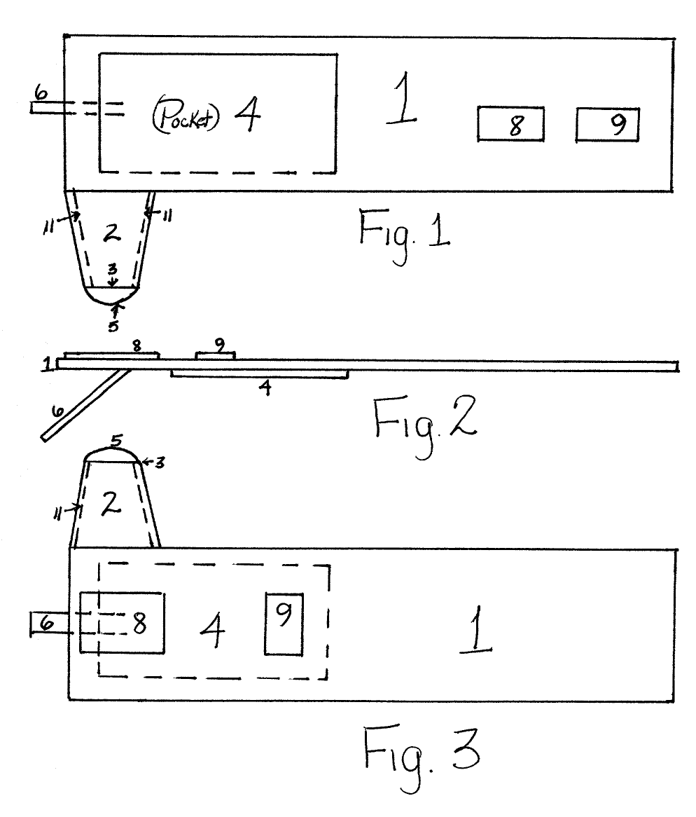

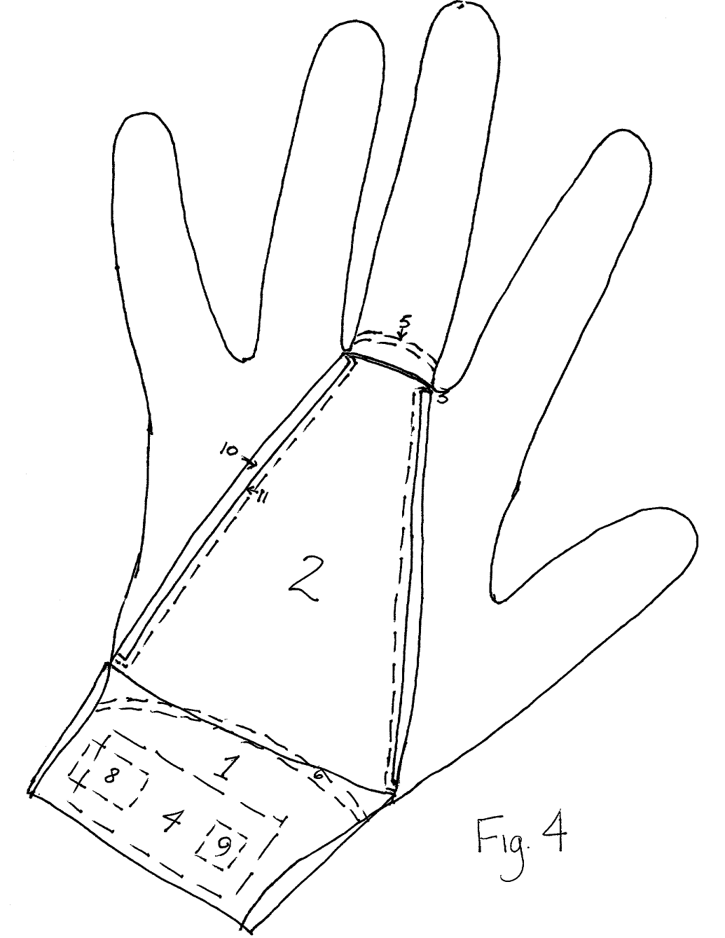

This device (100) also has fastening means (7) that is illustrated in FIGS. 1-3 using Velcro strips (8 and 9). It should be understood that other fastening means that is conventional in the prior art can be used instead of Velcro strips. Other such conventional fastening means are tie straps, belts and buckles, magnetic fasteners, hooks, snaps, etc. Although there is no general requirement that the fastening means be permanently attached to the outside of the piece (1), it is preferred that for embodiments designed for thermal treatment of the wrist that the fastening means be securely attached to the outside of the rectangularly shaped piece (1) for adjustment for comfort and appropriate fit.

Bones (10) can be inserted in pocket (11), especially designed for them at the side edges of the triangularly shaped piece (2). These bones are made out of a rigid material such as a strong plastic, wood, or metal and are used for immobilizing the wrist by adding rigid support.

In one embodiment, the components for the wrist wrap device (100) can be made of a soft elastic bandage type of material that is approximately 3 inches wide, two small covered elastic bands, a hot/cold pack, Velcro strips and two removable plastic bones that can be inserted to stabilize the range of motion. Although an elastic type of material is preferred, any type of material that is breathable can be used such as synthetic materials (e.g., rayon, polyamides, polyesters, and polypropylene) or materials made out of any natural fabric (e.g. cotton and wool). The only requirements of the fabric are that it be breathable yet be strong and contours with the hand and wrist and moves therewith without causing chaffing. The wrap can be worn with or without the removable plastic bones or ice or heat pack. The wrap can also be worn with the triangular support (2) placed over the back of the hand or over the palm of the hand for additional support for carpal tunnel sufferers. The elastic band (5) at the tip of the triangular support area (3) is designed to be placed over any finger of the wearer to further aid in the wearer's comfort. The hot/cold pack can be used as necessary.

The hot/cold pack is a conventional product in the market places that is well known in the medical and sporting industries. This hot/cold pack can be two separate units or a single unit. The single hot/cold pack is more preferred because it is relatively small for inserting in the wrap pocket (4) for providing heat or cold to a person's wrist. These hot/cold packs can contain a synthetic material that retains a great amount of heat for a sufficient time to treat an injured wrist. They can be heated in hot boiling water or placed in a microwave oven for a sufficient time to reach the desired temperature. The same material can be also refrigerated to get to low temperatures, including below freezing, for treating the wrist with cold.

The temperature of the present therapeutic thermal wrap device (100) is easily adjusted to therapeutically effective temperatures, and further, is able to maintain the therapeutically effective temperature for a period of time sufficient for treating an injured wrist with either heat or cold. The present therapeutic thermal wrap device after prior cooling to −1.5 degrees F. has been proven to maintain a temperature of less than 40° F. for seventeen hours in an 86° F. incubator. It is thought that 40° F. or less is a therapeutically effective temperature for cryotherapy. Minnesota Mining and Manufacturing Company, St. Paul, Minn. and Becton Dickinson and Company, Franklin Lakes, N.J. are examples of companies that manufacture such hoticold packs that can be used herein.

The long maintenance of the therapeutic effectiveness displayed by the present device, as well as the compact design, enables and greatly enhances portability. The case of portability of the present device is an unexpected advantage over prior thermal wraps. For example, the user can transport the present therapeutic thermal wrap to remote exercise sites over the course of several hours, participate in physical activities over the next several hours, then use the present wrap immediately after the physical activity while the wrap is still at a therapeutically effective temperature. The user could conceivably take the present device to work, leave the device in a car, locker or desk all day; then heat or cool the pack to the desired temperature, participate in up to 2-3 hours of exercise, and still be able to use the present wrap while it is still at an effective temperature level for thermotherapy or cryotherapy.

The present invention can be reused many times over a long period of time, and users will be able to rely on it as an easy, compact, rugged, inexpensive, and reliable means for dealing with wrist discomfort and other similar injuries.

The wrist wrap of the present invention is made from material that is cut and sewn into place. The bandage is grasped at one end and folded into a triangular shape and pinned, then stitched into place. The bandage is then folded over itself across the base of the triangle and pinned. It is then rolled out to a length of 24 inches and cut. That material is folded back against itself, doubling the thickness and pinned.

A second length of material approximately six inches long is cut and pinned to the underside of the wristband portion of the wrap (at the triangle end), thus creating a pocket for the hot/cold pack. That pocket section is stitched to the wristband, with the exception of a four-inch space along the top edge to allow for the insertion of the hot/cold pack.

A 2-inch length of the covered elastic band is fashioned into a loop and stitched to the underside of the uppermost comer of the triangular section of the wrap. The two lengths of plastic boning are provided for the user to insert into the pockets in the sides of the triangular sections as necessary. A second loop created from approximately 6 inches of the covered elastic band is stitched to the underside of the wristband section of the wrap, centered directly below the triangular section.

The loop sides of two one and one half inch Velcro strips are stitched lengthwise vertically at the center of the underside of the wristband, respectively, a triple width of the catch side of Velcro strips is horizontally stitched to the topside of the wristband area, centered below the triangle area, approximately one fourth inch from the edge. Spaced about one and three-fourth inches beyond is a second catch side of Velcro strip, stitched vertically to the center of the wristband. All folded and pinned areas are stitched in placed.

Although a description has been set forth above with dimensions, it should be understood that this invention could be made in sizes to accommodate different size wrists, i.e., from a child to a big and/or tall man or woman.

While the invention has been described with respect to specific embodiments, it should be understood that they are not intended to be limiting and that many variations and modifications are possible without departing from the scope and spirit of the invention.

What is claimed:

1. A therapeutic wrist wrap device comprising a triangularly shaped piece of material having a truncated apex with an elastic piece of material attached to the truncated apex at its corners and a base that is attached to a rectangularly shaped piece of material at one end on the longest dimensional side where the rectangularly shaped piece of material is of a sufficient length to completely encircle a person's wrist, has a smaller piece of material attached to the rectangularly shaped piece on three sides thereof to form a pocket on one side thereof for receiving either a heating pack or a cold pack therein, and attaching means for securing the device around a person's wrist, and an elastic band attached to the base of the triangularly shaped piece of material.

2. The therapeutic wrist wrap device of claim 1, wherein the triangularly shaped piece of material has pockets on two of its sides adjacent to its base for receiving immobilization means for keeping a person's wrist stable.

3. The therapeutic wrist wrap device of claim 1, wherein the immobilization means are slats or bones.

4. The therapeutic wrist wrap device of claim 1, wherein the heating and cold pack is a single unit that fits in the pocket on the rectangularly shaped piece of material.

5. The therapeutic wist wrap device of claim 1, wherein the wrist wrap is made from a synthetic or natural fabric material.

6. The therapeutic wrist wrap device of claim 5, wherein the synthetic fabric is selected from the group consisting of rayon, polyamide, polyester, and polypropylene.

7. The therapeutic wrist wrap device of claim 5, wherein natural fabric is selected from the group consisting of cotton and wool.

8. The therapeutic wrist wrap device of claim 1, wherein the device is sized as small, medium or large.

9. The therapeutic wist wrap device of claim 1, wherein the attaching means is selected from the group consisting of Velcro strips, belts and buckles, tie straps, magnetic fasteners, hook, and snaps.

10. The therapeutic wrist wrap device of claim 9, wherein the attaching means is Velcro strips.
